(19) United States
(12) Patent Application Publication
(10) Pub. No.: US 2013/0177977 A1
(43) Pub. Date: Jul. 11, 2013

(54) Systems and Devices for Restraining a Cell and Associated Methods

(71) Applicants: Quentin T. Aten, Orem, UT (US); Walter Fazio, Albuquerque, NM (US); Jason Mathew Lund, Orem, UT (US); Gregory H. Teichert, Provo, UT (US); Sandra H. Burnett, Saratoga Springs, UT (US); Brian D. Jensen, Orem, UT (US); Larry L. Howell, Orem, UT (US)

(72) Inventors: Quentin T. Aten, Orem, UT (US); Walter Fazio, Albuquerque, NM (US); Jason Mathew Lund, Orem, UT (US); Gregory H. Teichert, Provo, UT (US); Sandra H. Burnett, Saratoga Springs, UT (US); Brian D. Jensen, Orem, UT (US); Larry L. Howell, Orem, UT (US)

(73) Assignee: Brigham Young University, Provo, UT (US)

(21) Appl. No.: 13/539,184

(22) Filed: Jun. 29, 2012

Related U.S. Application Data
(60) Provisional application No. 61/502,617, filed on Jun. 29, 2011.

(51) Int. Cl. C12M 1/12; C12N 15/89

(52) U.S. Cl. CPC C12M 25/00; C12N 15/89
USPC 435/325; 435/283.1

(57) ABSTRACT

Systems, devices, and methods for restraining a biological structure are provided. In one example, a biological structure restraining device can include a barrier structure, an opening defined in the barrier structure, and at least two contact points positioned adjacent to the opening and oriented to contact the biological structure, wherein the barrier structure and the opening are structurally positioned to receive the cell at the contact points. In another aspect, the device can also include a biological structure manipulator having a structure operable to press the biological structure against the contact points. In yet another aspect, the device can further include a biological structure injector having a structure operable to be inserted through the opening and into the biological structure, wherein the biological structure manipulator is operable to maintain the biological structure against the contact points as the biological structure injector is inserted into the biological structure.

### PRIORITY DATA

This application claims the benefit of U.S. Provisional Patent Application Ser. No. 61/502,617, filed on Jun. 29, 2011, which is incorporated herein by reference in its entirety.

### BACKGROUND OF THE INVENTION

Microinjection of foreign materials into a biological structure such as a living cell can be problematic. Various transfection techniques include the microinjection of foreign genetic material such as DNA into the nucleus of a cell to facilitate the expression of foreign DNA. For example, when a fertilized oocyte (egg) is transfected, cells arising from that oocyte will carry the foreign genetic material. Thus in one application, organisms can be produced that exhibit additional, enhanced, or repressed genetic traits.

In some cases, researchers have used microinjections to create strains of mice that carry a foreign genetic construct causing macrophages to auto-fluoresce and undergo cell death when exposed to a certain drugs. Such transgenic mice have since played roles in investigations of macrophage activity during immune responses and macrophage activity during tumor growth.

Prior art microinjectors function in a similar manner to macro-scale syringes: a pressure differential forces a liquid through a needle and into the cell. In some cases a glass needle that has been fire drawn from a capillary tube can be used to pierce the cellular and nuclear membranes of an oocyte. Precise pumps then cause the expulsion of minute amounts of genetic material from the needle and into the cell. Researchers have produced fine microinjection needles made from silicon nitride and silica glass that are smaller than fire drawn capillaries. These finer needles generally also employ macro-scale pumps similar to those used in traditional microinjectors.

### SUMMARY OF THE INVENTION

The present disclosure provides systems, devices, and methods for restraining a biological structure. In one aspect, for example, a biological structure restraining device can include a barrier structure, an opening defined in the barrier structure, and at least two contact points positioned adjacent to the opening and oriented to contact a biological structure, wherein the barrier structure and the opening are structurally positioned to receive the biological structure at the contact points. In another aspect, the device can also include a biological structure manipulator having a structure operable to press the biological structure against the contact points. In yet another aspect, the device can further include a biological structure injector having a structure operable to be inserted through the opening and into the biological structure, wherein the biological structure manipulator is operable to maintain the biological structure against the contact points as the biological structure injector is inserted into the cell. In a further aspect, the device can include an angled recess around the opening, where the angled recess has a structural configuration to increase retention of the biological structure against the contact points.

In another aspect, the present disclosure provides a method of introducing biological material into a biological structure. Such a method can include positioning a biological structure against a barrier structure, the barrier structure having an opening there through, and pressing the biological structure against contact points adjacent to the opening with a biological structure manipulator. The method can also include inserting a biological structure injector having associated biological material through the opening and into the biological structure in a direction opposite the cellular manipulator, releasing the biological material from the biological structure injector, and withdrawing the biological structure injector from the biological structure such that the contact points are operable to reduce deformation of the biological structure.

### DEFINITIONS OF TERMS

In describing and claiming the present invention, the following terminology will be used in accordance with the definitions set forth below.

The singular forms “a,” “an,” and, “the” can include plural referents unless the context clearly dictates otherwise. Thus, for example, reference to “a support” can include reference to one or more of such supports, and reference to “an oocyte” can include reference to one or more of such oocytes.

As used herein, the term “biological material” can refer to any material that has a biological use and can be delivered into a biological structure. As such, “biological material” can refer to materials that may or may not have a biological origin. Thus, such material can include natural and synthetic materials, as well as chemical compounds, dyes, and the like.

As used herein, the term “biological structure” can refer to any structure having a biological origin. Biological structures can include single cell and multicellular structures.

As used herein, the term “injector” refers to any structure or device that can be utilized to introduce biological material into a biological structure. Non-limiting examples of biological structure injectors can include micropipettes, lances, and the like. As such, “injection” as used herein can include any technique for introducing a biological material into a biological structure that involves a biological structure injector.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint without affecting the desired result.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### BRIEF DESCRIPTION OF THE DRAWINGS

FIG. 1 shows a restraining device in accordance with one embodiment of the present invention.

FIG. 2 shows a restraining device in accordance with another embodiment of the present invention.

FIG. 3A shows a restraining device in accordance with another embodiment of the present invention.

FIG. 3B is an optical image of a restraining device in accordance with another embodiment of the present invention.

FIG. 4 shows a restraining device in accordance with another embodiment of the present invention.

FIG. 5 shows a restraining device in accordance with another embodiment of the present invention.

FIG. 6 shows a restraining device in accordance with another embodiment of the present invention.

### DETAILED DESCRIPTION

The present disclosure provides methods, devices, and associated systems for restraining a biological structure. In one aspect, the biological structure can be restrained during the delivery of a biological material thereinto. Traditional techniques of merely holding a cell with a suction pipette during such delivery can be problematic due to microscopic limitations in a three dimensional environment, alignment of a delivery device with the cell, movement of the cell during delivery, deformation of the cell during withdrawal of the injector, and the like. Additionally, traditional injection procedures are highly technical and time consuming, and often injection technicians need extensive training to become proficient. The often difficult techniques of such delivery procedures can be simplified by adequately restraining the cell (or biological structure) in a known position. It should be noted, however, that the present restraint techniques and structures can be utilized to restrain a biological structure for numerous reasons, and any reasoning for biological structure restraint is considered to be within the present scope.

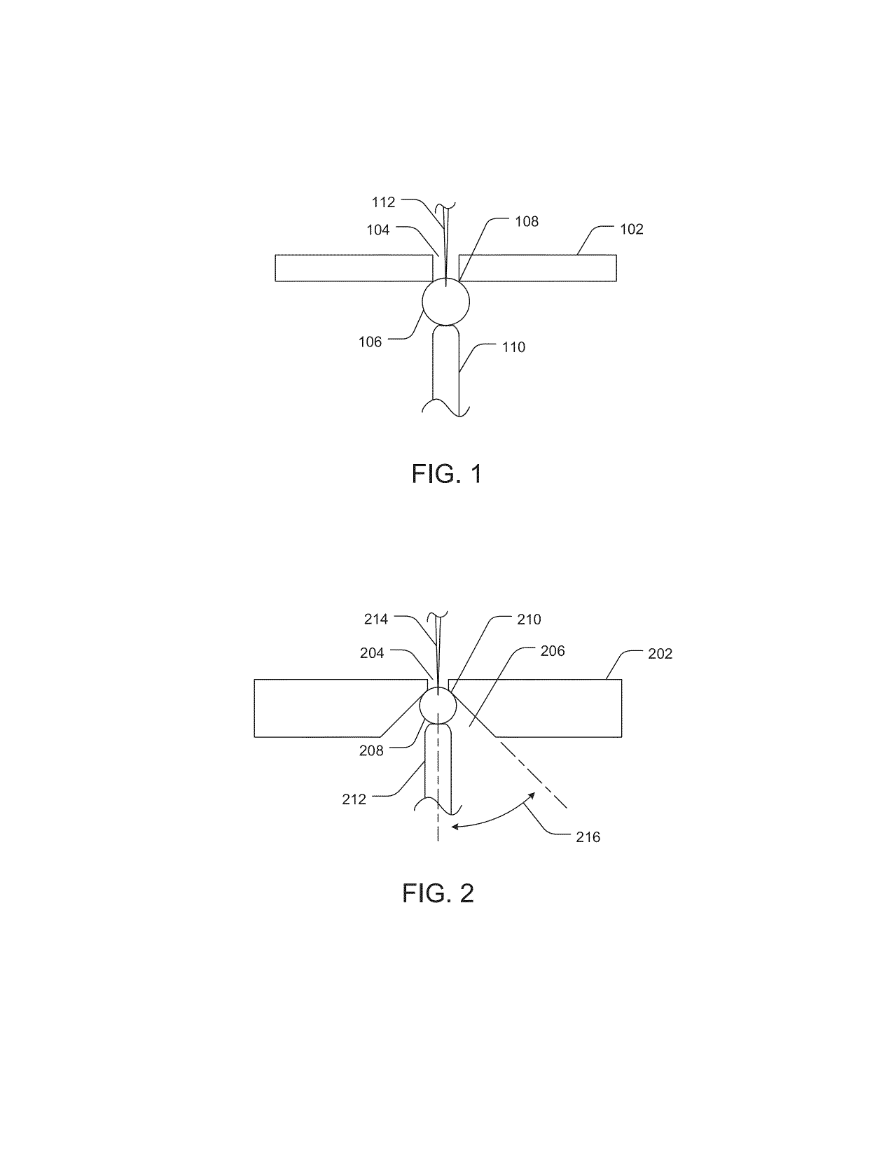

In one aspect, as is shown in FIG. 1 for example, a barrier structure 102 can have an opening 104 located there through. A biological structure, in this case a cell 106, is shown positioned adjacent to and contacting the barrier structure 102 at one or more contact points 108. In this case, the cell 106 is shown contacting the barrier structure 102 at two contact points 108 adjacent to the opening 104. The cell 106 can be held against the contact points 108 of the barrier structure 102 by pressing with a biological structure manipulator 110. Once the cell 106 is in position against the barrier structure 102, a biological structure injector 112 can be inserted through the opening 104 and into the cell 106. A biological material can be associated with the biological structure injector. Pressure applied by the biological structure manipulator 110 thus maintains the position of the cell 106 relative to the opening 104 during the procedure. Following the delivery of biological material, the biological structure injector 112 is withdrawn from the cell 106. In many traditional techniques the cell can be deformed and possibly damaged during withdrawal of a delivery apparatus. Cellular membrane and other cellular components can become associated with the injector, thus pulling away from the center of the cell during withdrawal. In the present example, the position of the cell 106 against the contact points 108 allows the biological structure injector 112 to be withdrawn with reduced cellular deformation. Thus as the biological structure injector 112 is withdrawn, the force applied by the contact points 108 can overcome the adherence forces between the cellular membrane and the biological structure injector, thus reducing cellular deformation. It should be noted that, in some aspects, a probe can be similarly utilized in place of the injector, as is described more fully below.

Such a barrier structure can thus restrain a biological structure such as a cell during cellular injection. Such restraining technique can greatly simplify the injection procedure, thus in some cases decreasing the time required to perform an injection and reducing many of the technical barriers associated with such procedures.

One of the technical difficulties associated with cellular injection involves the reorienting of a cell into a desired orientation. This difficulty would also be present with injections of biological material into multicellular structures. In many cases, experienced injection technicians can release and reapply suction from a holding pipette in a manner that allows a cell to roll over in the fluid media, thus facilitating reorientation. Such a technique, however, can be difficult to master, and can increase the time required for each injection. In one aspect of the present disclosure, the biological structure can be reoriented by rolling or otherwise manipulating the biological structure against the barrier structure. Such a reorienting can be done quickly and with minimal training Furthermore, in some aspects a structure can be provided for reorienting the biological structure that is distinct from the barrier structure.

The barrier structure can be made of a variety of materials and can have a variety of structural configurations. Any such material or configuration that allows restraint of a biological structure is considered to be within the present scope. Non-limiting examples of materials that can be used include semiconductors, ceramics, carbon nanotubes, glass, polymeric materials, metals, and the like, including combinations thereof. In one specific aspect, for example, the barrier structure can be made from an epoxy-based photoresist such as SU-8. Additionally, the barrier structure can be an extension of the material of the underlying substrate or it can be a separate material. A separate material can be formed on the underlying substrate, or it can be formed apart from the substrate and later associated therewith.

The physical configuration of the barrier structure can vary depending on the specifics of the biological structure being restrained, the equipment being used, and/or the preferences of the user. In some cases, for example, the physical configuration of the barrier structure can be designed to correspond to a particular biological structure type being restrained. For example, the opening in the barrier structure can vary depending on the size of the biological structure and/or the size of the biological structure injector that will pass there through. It can be beneficial, however, if the size of the opening is small enough to preclude the biological structure from passing there through or getting stuck therein. Thus the size of the opening can vary depending on various factors, and as such, should not be seen as limiting. In one aspect, however, the opening can be from about 25 microns to about 75 microns wide. In another aspect, the opening can be from about 50 microns to about 70 microns wide. In yet another aspect, the opening can be from about 60 microns to about 80 microns wide. In one specific aspect, the opening can be approximately 65 microns wide. In another specific aspect, the opening can be approximately 85 microns wide. Additionally, the opening can be of any physical configuration, such as circular, elliptical, polygonal, etc. In some aspects, the shape of the opening can be designed to further restrain the biological structure. Furthermore, in some aspects the opening can be a slot in the barrier structure. In one specific aspect, the opening can be configured to receive and/or allow the passage through, of a biological structure injector.

Additionally, the barrier structure can be of any height sufficient to restrain a biological structure. Thus the height of the barrier structure can vary depending on the size and shape of the biological structure being restrained. For example, a useful height may be about 100 microns. Such a barrier structure may adequately restrain biological structures having a size less than about 150 or 100 microns. In many cases, a barrier structure can restrain biological structures that have a midline that is less than the height of the barrier structure.

Various biological structure manipulator devices are contemplated, and any device capable of delivering and/or holding the biological structure in position at the opening of the barrier structure is considered to be within the present scope. In one aspect, for example, the biological structure manipulator can be a suction pipette. In another aspect, the biological structure manipulator can be a glass or polymeric rod. Additionally, one or more biological structure manipulators can be used. The biological structure manipulator(s) can be located and oriented in any position capable of maintaining the position of the biological structure during delivery of the biological material. Such positioning can also vary depending on the configuration of the barrier structure and the number of biological structure manipulators being used. In yet another aspect, the surrounding fluid media can be used as the biological structure manipulator, and the pressure holding the biological structure in position can be a positive pressure in the media applied from the side of the biological structure.

Various biological structure injector configurations are contemplated, and any such configuration is considered to be within the present scope. In one aspect, for example, the biological structure injector can be a traditional or nontraditional micropipette. Micropipettes can be made from a variety of materials, including various types of glass (e.g. borosilicate, aluminosilicate, etc.), quartz, polymers, ceramics, and the like. In the case of a glass micropipette, for example, the ends of a glass capillary tube can be pulled in opposite directions following the heating of a center region in order to create a micropipette having a sharp tip and a hollow interior. A micropipette can be filled with a solution containing a biological material to be injected into a biological structure. The micropipette is often coupled to a movement system such as a micromanipulator to allow precise movements of the tip of the micropipette. Thus, the micropipette is inserted into a biological structure, and the biological material is then expelled from the interior of the micropipette and into the biological structure. Although any technique for expelling the biological material is contemplated, in some aspects a micropump can be used. In other aspects, an electrical charge can be used to expel the biological material. Following delivery of the biological material, the micropipette can then be withdrawn from the biological structure.

In another aspect, the biological structure injector can be a lance. A lance is a solid or semisolid structure, and in some cases can have an internal channel. It is contemplated that a lance can be an integral part of a lance manipulation system, or the lance can be fabricated and utilized in traditional manipulation systems such as micromanipulators and the like. As such, in some aspects the lance is manufactured as a “stand alone” lance, and is not constrained to a fixed substrate upon which the lance was fabricated. Any size and/or shape of lance capable of delivering biological material into a biological structure is considered to be within the present scope. The size and shape of the lance can also vary depending on the biological structure receiving the biological material. The effective diameter of the lance, for example, can be sized to maximize survivability of the biological structure. It should be noted that the term “diameter” is used loosely, as in some cases the cross section of the lance may not be circular. Limits on the minimum diameter of the lance can, in some cases, be a factor of the material from which the lance is made and the manufacturing process used. In one aspect, for example, the lance can have a tip diameter of from about 5 nm to about 3 microns. In another aspect, the lance can have a tip diameter of from about 10 nm to about 2 microns. In another aspect, the lance can have a tip diameter of from about 30 nm to about 1 micron.

In a further aspect, the lance can have a tip diameter that is less than or equal to 1 micron. As such, in many cases the tip diameter of the lance can be smaller than the resolving power of current optical microscopes, which is approximately 1 micron.

Various lance materials are contemplated for use in constructing the lance, and any material that can be formed into a lance structure and is capable of carrying a charge is considered to be within the present scope. Non-limiting examples of lance materials can include a metal or metal alloys, conductive glasses, polymeric materials, semiconductor materials, carbon nanotubes, and the like, including combinations thereof. In one aspect, a lance can be a carbon nanotube or array of carbon nanotubes filled with a material such as carbon, silicon, and the like. Non-limiting examples of metals can include indium, gold, platinum, silver, copper, palladium, tungsten, aluminum, titanium, and the like, including alloys and combinations thereof. Polymeric materials that can be used to construct the needle structure can include any conductive polymer, non-limiting examples of which include polypyrrole doped with dodecyl benzene sulfonate ions, SU-8 polymer with embedded metallic particles, and the like, including combinations thereof.

In one exemplary use of such a lance, a nanoinjection procedure can be performed to introduce biological material into a biological structure. A lance and biological material can be brought into proximity outside of a biological structure. The lance can be positively charged and brought into contact with the biological material, which is accumulated at the tip portion of the lance. For example, a positive charge on the lance causes a negatively charged biological material to associate with and accumulate at the tip. A return electrode is placed in electrical contact with the medium surrounding the lance in order to complete an electrical circuit with a charging device. In the case of a cell, for example, the lance is then inserted through the cell membrane and into the cell. In some aspects, the lance is inserted through an opening in a barrier structure as is shown in FIG. 1. Biological material associated with the tip portion is inserted into the cell along with the lance. It is also contemplated that other techniques of associating the biological material with the lance in addition to electrostatic association are considered to be within the present scope. The lance is then discharged to allow the release of at least a portion of the biological material, which is thus delivered into the cell. Following release of the DNA, the lance can be withdrawn from the cell.

As has been described, in some aspects a probe can be utilized in place of the biological structure injector. In these cases, a biological structure can be restrained and then then probed using such a device. The materials, structure, and configuration of the probe can vary depending on the intended use of the device. For example, in one aspect the probe can be an electrode similar in nature to the lance, but rather than injection could be used for membrane potential measurements and/or experiments. One specific example would be a patch clamp device. In another aspect, the probe can be used for selective ablation of cells or cellular regions in a biological structure such as an embryo.

The length of a biological structure injector can be variable depending on the design and desired attachment of the biological structure injector to a movement system and the thickness and configuration of the barrier structure. In general, a biological structure injector has a length that is sufficient to pass through the opening in the barrier structure and penetrate a sufficient distance into the biological structure. Thus the length of the biological structure injector can be any length useful for a given delivery operation.

Further exemplary details regarding biological structure injectors, charging systems, movement systems, and biological structure restraining systems can be found in U.S. patent application Ser. Nos. 12/668,369, filed Sep. 2, 2010; Ser. No. 12/816,183; filed Jun. 15, 2010; 61/380,612, filed Sep. 7, 2010; and 61/479,777, filed on Apr. 27, 2011, each of which is incorporated herein by reference.

Biological material can be delivered into a variety of types of biological structures. In one aspect, for example, the biological structure can be a single cell. In another aspect, the biological structure can be multicellular. In yet another aspect, the biological structure can be an embryonic cell. In a further aspect, the biological structure can be a plurality of embryonic cells. Furthermore, both prokaryotic and eukaryotic cells are contemplated to receive biological material, including cells derived from, without limitation, mammals, plants, insects, fish, birds, yeast, fungus, and the like. Additionally, cells can include somatic cells or germ line cells such as, for example, oocytes and zygotes. The enhanced survivability of cells with the present techniques can allow the use of cells and cell types that have previously been difficult to microinject due to their delicate nature.

Additionally, various types of biological materials are contemplated for delivery into a biological structure, and any type of biological material that can be delivered into a biological structure is considered to be within the present scope. Non-limiting examples of such biological materials can include DNA, cDNA, RNA, siRNA, tRNA, mRNA, microRNA, peptides, synthetic compounds, polymers, dyes, chemical compounds, organic molecules, inorganic molecules, cells, and the like, including combinations thereof. In one aspect, the biological material can include DNA, cDNA, RNA, siRNA, tRNA, mRNA, microRNA, and combinations thereof. In another aspect, the biological material can include DNA and/or cDNA. Furthermore, in some aspects the biological material can be whole cells that are delivered into a biological structure. For example, embryonic stem cells can be delivered into an embryo or other multicellular biological structure. As one specific non-limiting example, whole cells can be injected into a blastocyst.

FIG. 2 shows a barrier structure 202 having an opening 204 extending through the barrier structure. An angled recess 206 is formed around the opening 204 to increase the retention of the biological structure (cell 208) against the contact points 210. The angled recess thus allows increased retention of the cell 208 at the opening 204, in some cases via force applied by the biological structure manipulator 212 pressing the cell 208 against the contact points 210. Once the cell is in position, a biological structure injector 214 can be used to penetrate the cell through the opening 204 in a direction opposite to the force applied by the cellular manipulator 212. The angled recess 206 has a cut out angle as is shown at 216.

The cut out angle can be any angle useful for retaining and/or manipulating a biological structure. The angle can vary depending on the size and shape of the biological structure being restrained. For example, larger cells may be more easily restrained in an angled recess having a wider cut out angle. Preimplantation stage mammalian embryos, for example, tend to be large and highly susceptible to deformation, and in some situations can be challenging to restrain under traditional injection methods. In such cases, the cut out angle of the barrier structure can be designed to easily restrain such cells. Similarly, the cut out angle can be configured to restrain multicellular biological structures. Additionally, a biological structure can be reoriented along the angled recess by pressing the biological structure against the recess wall with the biological structure manipulator or some other implement. The pressure against the biological structure will cause a rolling motion of the biological structure, thus allowing reorientation. If the biological structure manipulator is a suction pipette, the biological structure can be manipulated into a desired orientation, suctioned onto the end of the pipette, and moved into position at the opening of the barrier structure. In other cases, the biological structure can be rolled into position at the opening and into the desired position. Accordingly, the cut out angle can vary depending on the biological structure being manipulated and /or restrained. In one aspect, however, the angle can be from about 10° to about 30°. In another aspect, the angle can be from about 15° to about 25°. In yet another aspect, the angle can be from about 20° to about 25°.

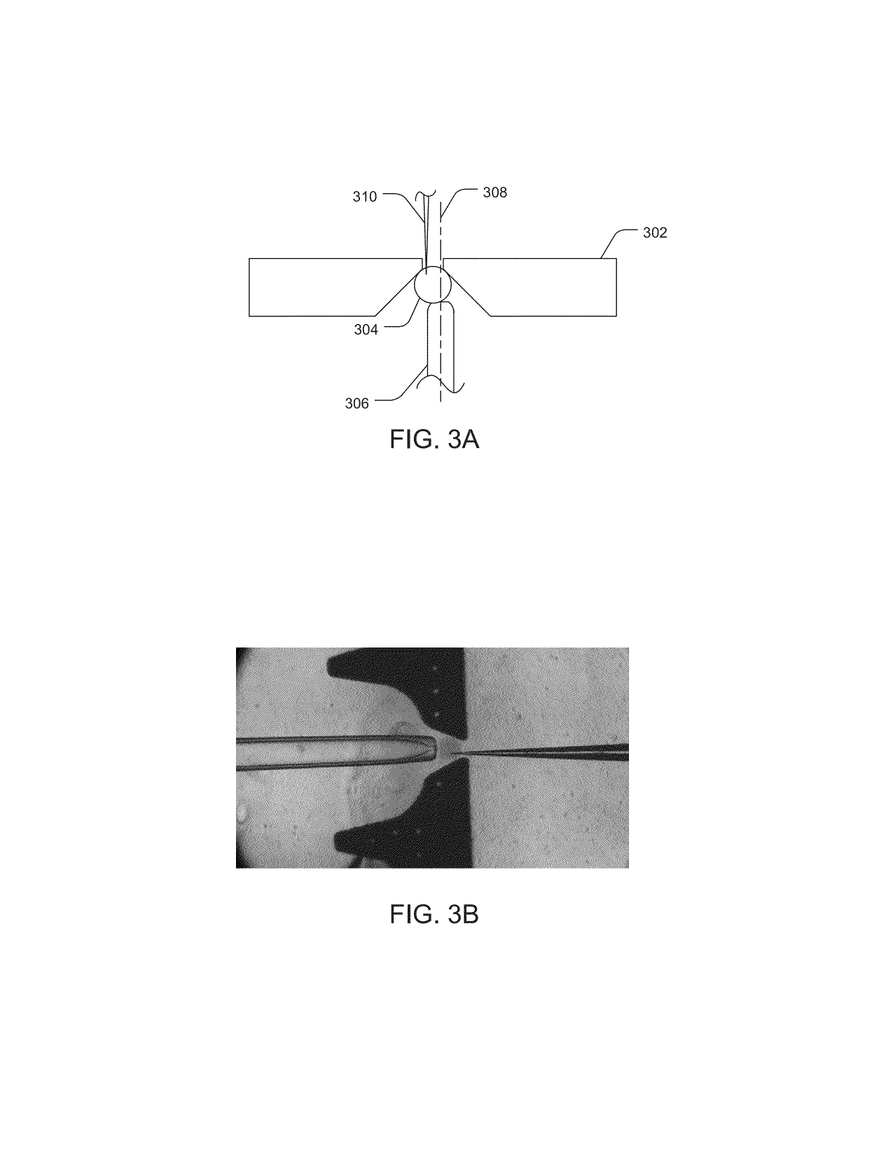

In another aspect, as is shown in FIGS. 3A and B, injection procedures can be implemented that can be very challenging with traditional microinjection technology. FIG. 3A shows a barrier structure 302 having an opening extending through the barrier structure and an angled recess formed around the opening as has been shown and described in FIG. 2. The angled recess allows increased retention of a cell 304 (or other biological structure) at the opening via force applied by the biological structure manipulator 306 pressing against the cell 304. In this case, the biological structure manipulator 306 is pressing against the cell 304 along an axis 308 that is offset from the centerline of the cell. Because the cell 304 is being held in the angled recess by the biological structure manipulator 306, a biological structure injector 310 can penetrate the cell opposite the direction of the applied force of the biological structure manipulator at an axial position that is offset from the axis 308 of the biological structure manipulator. FIG. 3B shows an optical image of an offset injection procedure. In traditional microinjection setups whereby only a suction pipette is used to hold the cell, attempting to penetrate the cell at an axial position that is offset from the axis of the suction pipette can cause the cell to move laterally with respect to the injection site, thus increasing the chance of damage to the cell. Thus the present barrier structure allows more flexibility in injection positions, thus potentially increasing the success of a procedure.

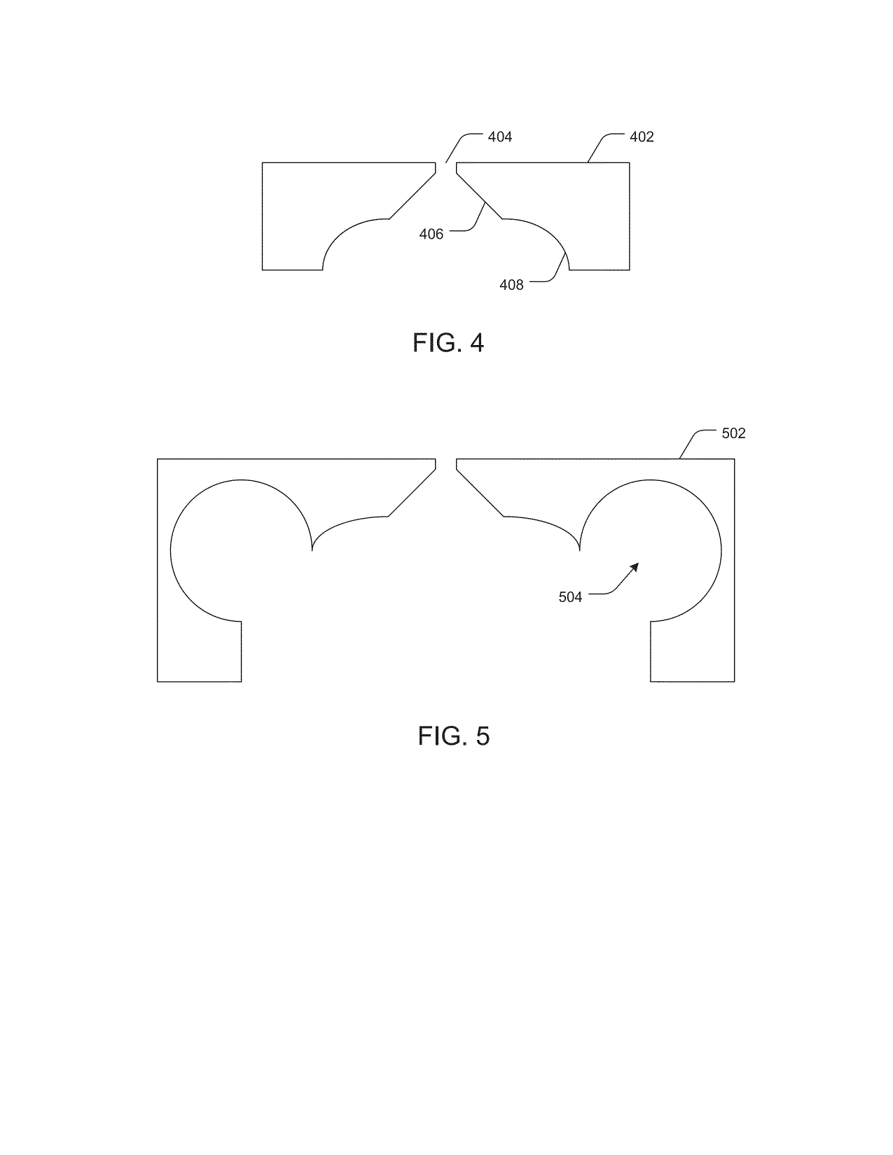

Additional structural implementations are also contemplated for use with the barrier structure. As is shown in FIG. 4, for example, a barrier structure 402 can include an opening 404 and an angled recess 406 around the opening 404. While the surface of the angled recess 406 can be used to reorient and otherwise manipulate a biological structure, additional structure having different physical configurations can be included for use as manipulating surfaces. Such a structure is shown at 408. As another example, FIG. 5 shows a barrier structure 502 having one or more cell sorting chambers 504. Such sorting chambers can allow cells to be more readily sorted and/ or kept track of during a procedure involving multiple cells.

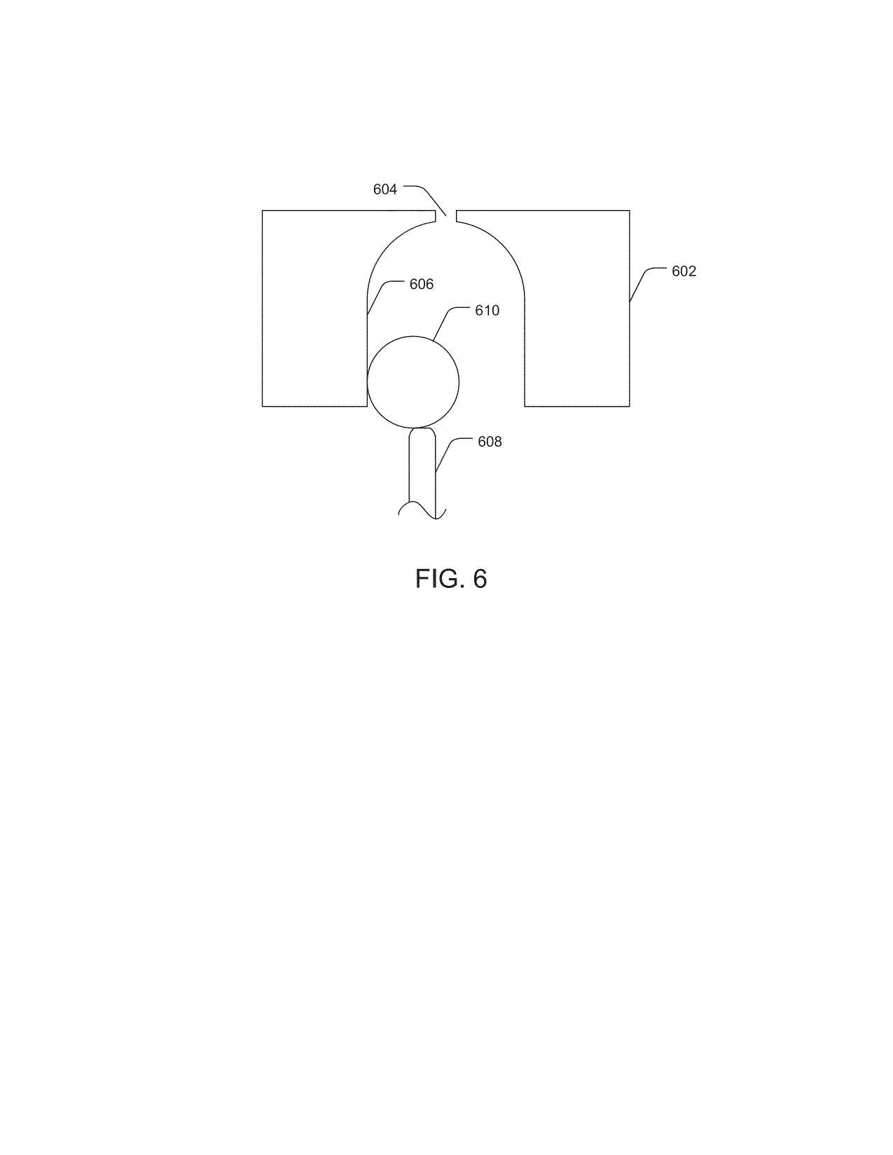

In another aspect, as is shown in FIG. 6, a barrier structure 602 can include an opening 604 and manipulating surface walls 606 forming a “U” shape and converging at the opening 604. In this case, a biological structure manipulator 608 can reorient the cell 610 along the manipulating surface walls 606 prior to pressing the cell against the opening 604.

In another aspect, the present disclosure provides a method of introducing biological material into a cell. Such a method can include positioning a cell against a barrier structure, the barrier structure having an opening there through, pressing the cell against contact points adjacent to the opening with a cellular manipulator, and inserting a lance having associated biological material through the opening and into the cell in a direction opposite the cellular manipulator. The method also includes releasing the biological material from the lance and withdrawing the lance from the cell such that the contact points are operable to reduce deformation of the cell.

### EXAMPLE

The following is an example of creating a cell manipulation structure.

A glass substrate is cleaned with acetone and isopropyl alcohol and dried with a nitrogen gun. The substrate is then O₂ plasma descum processed at 200 Watts for 3 minutes. A portion of SU-8 2025 resist is applied to the substrate, about 1 ml for every 25 ml of substrate diameter. The substrate is placed in a spinner and spun as follows:
Step 1: 500 rpm with 86 rpm/s acceleration for 6 seconds.
Step 2: 1000 rpm with 344 rpm/s acceleration for 40 seconds.
Step 3: 6000 rpm with 6020 rpm/s acceleration for 2 seconds

The substrate is placed on a hotplate at 65° C. for 2 minutes, followed by a ramp to 95° C. and a hold for 20 minutes. Following baking, the sample is cooled for at room temperature for 7 minutes.

The SU-8 is patterned with a dark field mask. As SU-8 is a negative resist, SU-8 exposed to UV light is crosslinked and remains on the substrate, while SU-8 that is not exposed to UV is removed. The substrate is exposed to 26.5 mW/cm² for 405 nm wavelength UV for 21 seconds.

The substrate is post-exposure baked on a hotplate at 65° C. for 2 minutes, then ramped to 95° C., with a hold for 5 minutes. The substrate is cooled at room temperature for 7 minutes.

The substrate is then placed in a dish containing SU-8 developer (Microchem), and gently agitated for 7 minutes, followed by cleaning with an acetone rinse, an isopropyl alcohol rinse, and a light drying with a nitrogen gun.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A device for restraining a biological structure, comprising:
a barrier structure;
an opening defined in the barrier structure; and
at least two contact points positioned adjacent to the opening and oriented to contact the biological structure, wherein the barrier structure and the opening are structurally positioned to receive the biological structure at the contact points.

2. The device of claim 1, wherein the barrier structure is coupled to a transparent substrate.

3. The device of claim 2, wherein the transparent substrate is a specimen slide.

4. The device of claim 2, wherein the barrier structure is formed on the transparent substrate.

5. The device of claim 2, wherein the barrier structure is formed from the transparent substrate.

6. The device of claim 1, further comprising a biological structure manipulator having a structure operable to press the biological structure against the contact points.

7. The device of claim 6, further comprising a biological structure injector having a structure operable to be inserted through the opening and into the biological structure, wherein the biological structure manipulator is operable to maintain the biological structure against the contact points as the biological structure injector is inserted into the biological structure.

8. The device of claim 7, wherein the biological structure manipulator is positioned to apply a force that is approximately opposite in direction relative to the insertion direction of the biological structure injector through the opening.

9. The device of claim 7, wherein the contact points restrain the biological structure from being deformed as the biological structure injector is withdrawn from the biological structure.

10. The device of claim 7, wherein the biological structure injector is a microinjection pipette.

11. The device of claim 7, wherein the biological structure injector is a lance.

12. The device of claim 6, wherein the biological structure manipulator is operable to reorient the cell by moving the biological structure along a portion of the barrier structure.

13. The device of claim 1, further comprising an angled recess around the opening, the angled recess having a structural configuration to increase retention of the biological structure against the contact points.

14. The device of claim 1, wherein the opening is configured to receive a microinjection pipette.

15. The device of claim 1, wherein the opening is configured to receive a lance.

16. A method of introducing biological material into a biological structure, comprising:
positioning a biological structure against a barrier structure, the barrier structure having an opening there through;
pressing the biological structure against contact points adjacent to the opening with a biological structure manipulator;
inserting a biological structure injector having associated biological material through the opening and into the biological structure in a direction substantially opposite the biological structure manipulator;
releasing the biological material from the biological structure injector; and
withdrawing the biological structure injector from the biological structure such that the contact points are operable to reduce deformation of the biological structure.

17. The method of claim 16, wherein the biological structure injector is inserted into the biological structure along an axis substantially offset from a centerline axis of the biological structure manipulator, and wherein the insertion of the biological structure injector does not cause a substantial lateral displacement of the biological structure.

18. The method of claim 16, wherein the biological structure is a cell.

19. The method of claim 16, wherein the biological structure is a multicellular structure.

20. The method of claim 16, wherein the biological structure is an embryonic cell.

21. The method of claim 16, wherein positioning the biological structure further includes moving the biological structure along the barrier structure to position the biological structure in a preferred orientation.
